(19) United States
(12) Patent Application Publication
(10) Pub. No.: US 2022/0251295 A1
(43) Pub. Date: Aug. 11, 2022

(54) LEVODOPA POLYMERIC CONJUGATES, FORMULATIONS THEREOF, AND THEIR USES FOR THE TREATMENT OF PARKINSON'S DISEASE

(71) Applicant: DYNAMIC BIOLOGICS INC., Monmouth Junction, NJ (US)

(72) Inventors: Deven Patel, Cedar Grove, NJ (US); H. Rajan Sharma, Belle Mead, NJ (US); Manoj Sharma, Woodstock, MD (US); Leema Reddy PEDDAREDDYGARI, Monroe, NJ (US)

(21) Appl. No.: 17/623,576

(22) PCT Filed: Jun. 28, 2020
(86) PCT No.: PCT/US20/40034

§ 371 Date: Dec. 28, 2021

Related U.S. Application Data
(60) Provisional application No. 62/868,134, filed on Jun. 28, 2019.

(51) Int. Cl. C08G 65/333; A61K 9/00; A61K 9/127; A61K 47/56; A61K 47/50; A61K 45/06

(52) U.S. Cl. CPC C08G 65/33303; A61K 9/0019; A61K 45/06; A61K 47/56; A61K 47/50; A61K 9/127

(57) ABSTRACT

Compounds of formula I: or a pharmaceutically acceptable salt, hydrate, and/or solvate thereof, wherein: R₁ is a pharmaceutically acceptable polymeric moiety comprising a pharmaceutically acceptable polymer chain such that the carbonyl group is linked to R₁ through an ester, amide, carbonate or carbamate bond; R₂ is hydrogen, or —(C═O)Rs wherein R₅ is a C₁₋₃ straight or branched chain alkyl group; and R₃ and R₄ are independently selected from hydrogen, C₁₋₃ straight or branched chain alkyl group, or —(C=0)Re wherein R₆ is —(O—CH₂—CH₂)ₙ—OCH₃ or a C1-3 straight or branched chain alkyl group, and n is 1 to 5. The compositions are useful for the treatment of Parkinson's disease when administered alone or in combination with carbidopa and/or entacapone.

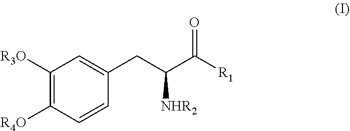

### CROSS REFERENCE TO RELATED PATENT APPLICATION

This application claims priority to provisional U.S. Application No. 62/868,134 filed Jun. 28, 2019, the contents of which are herein incorporated by reference in their entirety.

### FIELD OF INVENTION

The present invention relates to polymer conjugates of levodopa and its prodrugs, and polymeric nanoparticle/microparticle formulations of the polymer conjugates. These compounds and compositions are useful for the treatment of Parkinson's disease.

### BACKGROUND OF THE INVENTION

Levodopa is the common name for (S)-2-amino-3-(3,4-dihydroxyphenyl)propanoic acid, an

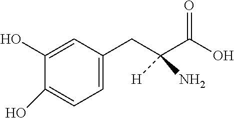 [SMILES: N[C@@H](Cc1ccc(O)c(O)c1)C(=O)O]

aromatic amino acid derivatives which is the main source of dopamine. In human and in other animals, levodopa is synthesized from amino acid L-tyrosine and serves as the precursor in the synthesis of neurotransmitters dopamine, norepinephrine (noradrenaline), and epinephrine (adrenaline), which are collectively known as catecholamines.

Parkinson's disease (PD) is a progressive neurodegenerative disease that affects approximately 1-2% of the population above the age of sixty. Symptoms include resting tremor, rigidity, slowness of movement and postural instability caused by selective degeneration of dopaminergic neurons in the substantia nigra leading to disruption of the nigrostriatal pathway and decreased striatal dopamine levels. Olanow et al., Neurology. 2009; 72(21 Suppl 4):S1-136.

The efficacy of high dose levodopa (3-16 g/day) in treating PD was first reported in 1969 (Cotzias et al., N. Engl. J. Med. 1969; 280(7):337-345; Yahr et al., Arch. Neurol. 1969; 21(4):343-354). The United States Food and Drug Administration (“FDA”) approved levodopa for treatment for PD in 1970. Levodopa, unlike dopamine can cross the blood brain barrier (BBB) and is converted to dopamine in the central nervous system as well as in the peripheral circulation. Most commonly, levodopa is used as a dopamine replacement agent for the treatment of PD and is particularly effective in controlling the bradykinetic symptoms that are apparent in PD. Levodopa is recommended for symptomatic treatment of all stages of Parkinson's disease and is given multiple times every day by oral route. Levodopa is commonly administered with carbidopa, a dopamine decarboxylase inhibitor, to decrease the amount of levodopa that is converted to dopamine in the periphery. This combination therapy allows for more levodopa to cross the BBB. Once converted to dopamine, it activates the postsynaptic dopaminergic receptors and compensates for the decrease in endogenous dopamine.

Levodopa is absorbed in the small bowel and 95% of an administered oral dose is pre-systemically decarboxylated to dopamine by the aromatic L-amino acid decarboxylase (AADC) enzyme in the stomach, lumen of the intestine, kidney, and liver. Levodopa may also be methoxylated by the hepatic catechol-O-methyltransferase (COMT) enzyme system to 3-Omethyldopa (3-OMD), which cannot be converted to central dopamine. Therefore, only a small portion of the oral dose of levodopa is transported across the BBB into the central nervous system (CNS) where it is converted to the neurotransmitter dopamine by the brain's AADC enzyme. Dopamine is further converted to sulfated or glucuronidated metabolites, and homovanillic acid through various metabolic processes. The primary metabolites of levodopa are 3,4-dihydroxyphenylacetic acid (13-47%) and homovanillic acid (23-39%).

Because gastric AADC and COMT enzymes degrade levodopa, the drug is given with:
i) a peripheral dopamine decarboxylase inhibitors (carbidopa), without which 90% of levodopa is metabolized in the gut wall, and
ii) a COMT inhibitor (entacapone), which prevents peripheral loss of levodopa about a 5%.

Inhibitors of AADC and COMT inhibit decarboxylation of levodopa in the stomach and periphery, making more levodopa available for transport across the BBB to increase the dopamine content of the brain. Carbidopa reduces the amount of levodopa required to produce a given response by 75% when administered with levodopa. Co-administered with levodopa/carbidopa, a 200 mg dose of entacapone increases levodopa plasma exposure by 35-40%.

Plasma half-life of levodopa alone is about fifty (50) minutes. When administered along with carbidopa (Sinemet® and Sinemet® CR 50-200), that half-life is increased to 1.5 hours (Sinemet® label, NDA17555). The time to reach peak plasma concentration (Tₘₐₓ) was about 0.5 hours for Sinemet® and 2 hours for Sinemet® CR, the peak plasma concentration (Cₘₐₓ) was 1151 ng/mL vs. 3256 ng/mL for Sinemet® vs Sinemet® CR (Sinemet® CR label, NDA 019856). Following administration of Stalevo® (carbidopa, levodopa and entacapone combination, 37.5/150/200 mg), the tₘₐₓ is about 1.5 hours and Cₘₐₓ is 1270±329 ng/mL (STALEVO® label, NDA 21485).

Common side effects of levodopa include nausea, vomiting, dry mouth, loss of appetite, heartburn, diarrhea, constipation, dizziness, muscle pain, numbness or tingly feeling and trouble sleeping. Serious side effects include mood changes, increased eye blinking/twitching and worsening of involuntary movements/spasms. Motor fluctuations, including dyskinesia and abnormal involuntary movements, are closely linked to the pharmacokinetics of levodopa, its irregular uptake, short half-life, low bioavailability and marked fluctuations in plasma concentrations. LeWitt, Mov. Disord. 2015; 30(1):64-72; Tambasco et al., Curr Neuropharmacol. 2018; 16(8):1239-1252.

Development of dyskinesia can be avoided by using lower doses of levodopa and by maintaining steady dopamine levels. Research is ongoing to find a delivery route for levodopa to achieve continuous dopaminergic stimulation. An intrajejunal infusion developed by Abbvie (Duopa®) is given by continuous infusion for the treatment of motor fluctuations in patients with advanced Parkinson's disease approved by FDA in 2015. A levodopa inhalation powder, Inbrija® by Acorda Therapeutics, Inc., was approved by the FDA in 2018. Some other formulations for continuous subcutaneous (SC) infusion, such as ABBV-951 (Abbvie) and ND6012 (Neuroderm/Mitsubishi Tanabe) are under development.

Levodopa has been modified to water soluble esters as well as amide derivatives for better brain uptake. Since there are three types of active functional groups in Levodopa to modify as prodrug derivatives, many prodrugs are reported. There are two benzylic hydroxyl groups at 3,4-position, one amine group at 2-position and one active carboxyl group at the terminal. The two hydroxyl groups of levodopa can be modified to ester derivatives. The methylester of Levodopa (Levomet®) is already in the market. However, the ethyl ester derivatives (Etilevodopa, TV-1203) was found to be less efficacious than Levodopa in Phase III clinical trials.

### SUMMARY OF THE INVENTION

The invention provides certain polymer conjugates of levodopa and its prodrugs with linear, branched and globular biocompatible polymers. These compounds offer sustained-release properties compared to free levodopa which has a very short half-life. The invention also provides nanoparticle/microparticle formulations of polymer conjugates of levodopa and its prodrugs using biocompatible pharmaceutically acceptable polymers. The compounds and compositions of the invention provide improved bioavailability and reduce the frequency of dosing and total dosage of levodopa, thereby improving the side effect profile of levodopa, used alone or in combination with carbidopa and/or entacapone.

In some embodiments, the present invention provides a compound of formula I:

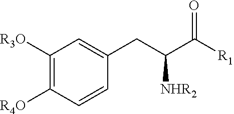

or a pharmaceutically acceptable salt, hydrate, and/or solvate thereof, wherein:
R₁ is a pharmaceutically acceptable polymeric moiety comprising a pharmaceutically acceptable polymer chain such that the carbonyl group is linked to R₁ through an ester, amide, carbonate or carbamate bond;
R₂ is hydrogen, or —(C═O)R₅ wherein R₅ is a C₁₋₃ straight or branched chain alkyl group; and
R₃ and R₄ are independently selected from hydrogen, C₁₋₃ straight or branched chain alkyl group, or —(C═O)R₆ wherein R₆ is —(O—CH₂—CH₂)ₙ—OCH₃ or a C₁₋₃ straight or branched chain alkyl group, and n is 1 to 5.

Yet other embodiments of the present invention provide a pharmaceutical composition comprising micro or nano particles comprising:
a pharmaceutically effective amount of the compound of formula I; and
a second pharmaceutically acceptable polymer,

wherein the compound of formula I is encapsulated in the second pharmaceutically acceptable polymer. Pharmaceutically acceptable polymers used in the present invention may be linear, branched or globular.

In some embodiments of the invention, the pharmaceutically acceptable polymer and/or the second pharmaceutically acceptable polymer is independently selected from the group consisting of polyethylene glycol (PEG), poly(glycolide) (PGA), poly(lactide) (PLA), poly(caprolactone), poly(lactide-co-caprolactone), poly(lactide-co-glycolide) (PLGA), and poly(lactic acid)-butanol, poly(vinyl pyrrolidone), poly(vinyl alcohol) (PVA), poly(ethyleneimine), poly(malic acid), poly(L-lysine), poly(L-glutamic acid), and poly ((N-hydroxyalkyl)glutamine), dextrins, hydroxyethylstarch, polysialic acid, polyacetals, N-(2-hydroxypropyl)methacrylamide copolymer, poly(amido amine) dendrimers, and mixtures, combinations and copolymers thereof. In some embodiments of the invention, the pharmaceutically acceptable polymer and/or the second pharmaceutically acceptable polymer used for encapsulation is selected from the group consisting of PLA, PGA, PLGA, PVA, and combinations thereof in different proportions.

Certain embodiments of the invention provide compositions comprising a pharmaceutically effective amount of the compound formula I and one or more pharmaceutically acceptable carriers or excipients. In particular, castor oil or its derivatives may be used as an excipient. In some embodiments, the compositions are in the form of liposomes or micelles using pharmaceutically acceptable amphiphilic compounds.

In certain embodiments of the invention, the micro or nano particles compositions comprising polymer encapsulated compound of formula I further comprise one or more pharmaceutically acceptable carriers or excipients.

The compositions of the invention are useful for treatment of PD. In particular, the compositions are useful for the same treatments as levodopa, used alone or in combination with carbidopa and/or entacapone, such as indications of levodopa alone or in combination with carbidopa and/or entacapone approved by USFDA or medicine regulatory agencies of other countries.

In some embodiments, the compositions of the invention may be administered by a parenteral route, such as intravenously, intramuscularly, intraperitoneally, or subcutaneously. In certain other embodiments, the compositions of the invention may be administered topically, such as in the form of transdermal patches, creams, foams, gels, lotions, ointments, sprays, and eye drops that are applied epicutaneously, applied to the conjunctiva or through inhalation.

In some embodiments, the compositions of the invention may be administered once daily, or twice or thrice weekly. In other embodiments, the compositions of the invention may be administered once weekly, biweekly, or once monthly.

The compositions of the invention offer improved chemical and pharmaceutical properties, such as superior pharmacokinetic properties, compared to levodopa and require substantially reduced dosage to achieve therapeutic plasma concentration due to the structure of the compound of formula I, the nature of the compositions, and/or the mode of administration. The compositions of the invention reduce adverse events and variability in pharmacokinetics.

### DETAILED DESCRIPTION

The prodrug of levodopa are obtained by using suitable chemical moieties which mask one or both reactive hydroxyl groups in the phenyl ring and/or the amine group of levodopa. In some embodiments, 0-diacetyl derivatives or a short poly ethylene glycol (PEG) unit (repeating unit n=1-5) at 3 and 4 position of levodopa can be employed generating an ester bond which is eventually converted to free Levodopa in the body system. The two hydroxyl groups can also be converted to 0-methoxy groups for prolonged duration of action. It has been established that the amide prodrug of Levodopa in the form of acetamide in which the amine group is converted to acetamide has better Cₘₐₓ, tₘₐₓ and AUC (the area under the curve describing the variation of a drug concentration in blood plasma as a function of time) as compared to Levodopa upon systemic administration (Jiang et al., J. Pharm. Biomed. Anal. 2010; 53:751-754). So, an N-acetylation reaction can be done with levodopa to employ an acetamide group for improved efficacy. It is worth noting here that, in the majority of prodrug formulations of levodopa, the Cₘₐₓ, AUC and tₘₐₓ values in the plasma are known. It is not necessary that a better Cₘₐₓ value in plasma of a particular prodrug formulation has better brain uptake. It has been proven that even if there is no difference in Cₘₐₓ and tₘₐₓ in plasma, an elevated amount of dopamine was observed in the brain with such prodrugs as compared to Levodopa (Ishikura et al., Int. J. Pharm. 1995; 116:51-63).

An in vivo cleavable bond is generated with the carboxylic acid functional group of levodopa to a biocompatible polymer so that the polymer allows the levodopa to circulate in blood plasma for longer time without clearance. It also reduces the chances of peripheral degradation of levodopa to dopamine by AADC and COMT enzymes, thereby increasing the subsequent availability of levodopa in the brain. The conjugated compound of formula I provides sustained plasma levels of levodopa with increased delivery of levodopa to the brain, resulting in improved efficacy.

Pharmaceutically acceptable polymers used in the present invention may be non-toxic, non-immunogenic, non-antigenic, highly soluble in water and FDA (The Food and Drug Administration) approved. The covalent attachment of polymer to a drug can increase its hydrodynamic size (size in solution), which prolongs its circulatory time by reducing renal clearance (Knop et al., Angew. Chemie Int. Ed. 2010; 49(36):6288-6308; Veronese et al., Drug Discov Today. 2005; 10(21):1451-1458; and Harris et al., Nat Rev Drug Discov. 2003; 2(3):214-221). The polymer conjugate compounds of the invention and polymer-encapsulated compositions of the invention have several advantages including increased bioavailability at lower doses; predictable drug-release profile over a defined period of time following each administration; better patient compliance; ease of application; improved systemic availability by avoidance of first-pass metabolism; reduced dosing frequency without compromising the effectiveness of the treatment; decreased incidence of side effects; and overall cost reduction of medical care.

Polymer conjugates of formula I may be prepared by methods known in the art, for example, Sk U H et al., Biomacromolecules. 2013; 14(3):801-810. Polymer-encapsulated micro/nano particles may be prepared by methods known in the art. For example, Han et al., Front Pharmacol. 2016; 7:185; Qutachi 0 et al., Acta Biomater. 2014; 10(12):5090-5098.

In some embodiments, the pharmaceutically acceptable polymer chain in compounds of formula I comprises 15-75 monomer units, 20-70 monomer units, or 25-65 monomer units. In other embodiments, the polymer has a molecular weight in the range of 1 kDa to 75 kDa, 2 kDa to 60 kDa, or 3 kDa to 50 kDa.

In certain other embodiments, the pharmaceutically acceptable polymer chain in the compound of formula I is a straight or branched chain PEG comprising 4-120 monomer units, 4-75 monomer units, 4-50 monomer units, or 4-30 monomer units. In certain other embodiments, the polymer is a straight or branched chain PEG comprising 12-120 monomer units, 12-75 monomer units, 12-75 monomer units, or 12-30 monomer units. In some other embodiments, the polymer chain is a straight or branched chain PEG comprising 11-20 monomer units, 26-42 monomer units, 49-64 monomer units, or 72-111 monomer units. In certain other embodiments, the polymer chain is a straight or branched chain PEG having a molecular weight in the range of 0.4 kDa to 50 kDa, 0.5 kDa to 50 kDa, 0.8 kDa to 50 kDa, or 1 kDa to 50 kDa.

The term “encapsulated” in the context of the present invention means coated by, covered by, or surrounded by, such that about 20% to about 80% of the compound of formula I is enclosed/covered/coated by the polymer.

In some embodiments, PLGA and mixture of PLGA with other polymers, such as PLA, PGA and PVA, in different ratios are used to encapsulate compounds of the invention to form microparticles. Due to its excellent biocompatibility, PLGA is a pharmaceutically acceptable biodegradable polymer widely used for encapsulation of a broad range of therapeutic agents including hydrophilic and hydrophobic small molecule drugs, DNA, and proteins. Other additives can be used to enhance the drug loading and efficiency in PLGA microparticles, such as PEG, poly(orthoesters), chitosan, alginate, caffeic acid, hyaluronic acid etc. PLGA can be a varying composition of PLA and PGA with a ratio from 20 to 80% PGA in PLA and vice versa.

In some embodiments, the amount of compound of formula I in the compositions of the invention is in the range of 100 mg to 2000 mg equivalent of levodopa administered once daily. Compositions comprising 10-200 mg of carbidopa and/or 200-1600 mg of entacapone may be used in combination with the compositions of the invention for treatment of PD. In some embodiments, compositions of the invention may include carbidopa and/or entacapone in addition to the compound of formula I. The amount of carbidopa co-administered with levodopa may be in a ratio of 1:10 to 1:4 with respect to the amount of levodopa. Entacapone may be co-administered with levodopa in a dose of 200 mg and the dosage repeated as required. Carbidopa in an amount of 10 mg to 200 mg/day and/or entacapone in an amount of 200 mg to 1600 mg/day may be co-administered with the compounds or compositions of the invention.

In some embodiments, dosage forms of the composition of the invention are adapted for administration to a patient parenterally, including subcutaneous, intramuscular, intraperitoneal, intravenous or intradermal injections. In other embodiments, the composition may be administered as a depot. Upon parenteral injection of levodopa polymer conjugates of formula I, enzymatic cleavage may occur generating levodopa and/or its prodrugs, and the respective polymer used in the conjugation.

In some embodiment, the compositions of the invention further comprise one or more pharmaceutically effective carriers or excipients. Pharmaceutical compositions adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents.

The compositions may be presented in unit-dose or multi-dose containers, for example, sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

### EXAMPLES

a) Preparation of Polymer Conjugates of Formula I

Levodopa may be prepared by methods known in the art or obtained from commercial sources. All prodrug of Levodopa (ester at 3,4-position and amide at 2-position) may also be prepared by methods known in the art.

Dissolve levodopa or its prodrug in anhydrous dimethylformamide (DMF) under nitrogen atmosphere. Add: N-(3-Dimethylaminopropyl)-N′-ethylcarbodiimide hydrochloride (EDC) and Dimethylamino) pyridine (DMAP) dissolved in DMF to the reaction mixture and stir the reaction mixture for 30 minutes. Add a calculated amount of linear, branched PEG or any other carboxylate-functionalized globular polymer dissolved in DMF to the reaction mixture and stir the reaction mixture for 2 days under nitrogen atmosphere. Evaporate the solvent and dialyze the resulting reaction mixture for 24 hours using dialysis membrane (MWCO 1 kDa) and then with water. Lyophilize the resulting water to get the final levodopa polymer conjugates. Check the purity of the conjugate by reverse-phase high performance liquid chromatography (HPLC) and characterize/calculate the loading of the polymeric conjugate by proton nuclear magnetic resonance (NMR), and matrix assisted laser desorption/ionization time-of-flight (MALDI-TOF) mass spectroscopy.

b) Preparation of Microparticles of Compound of Formula I

Nanoprecipitation technique is used for the preparation of the levodopa microparticles. Briefly, either levodopa or levodopa prodrug and a polymer (e.g., PLGA) are dissolved in a suitable solvent (e.g., dichloromethane) in different ratios, the mixture being subjected to sonication for 5-10 minutes to achieve dissolution, if required. Dissolve a hydrophilic non-ionic surfactant (for example a triblock copolymer), such as Pluronic F127, in 50 mL of deionized water and add the levodopa/PLGA solution dropwise using a syringe with a flow rate of 1 mL/10 min with stirring at varying speed. Centrifuge, and lyophilize the obtained nanosuspension with cryoprotectant (e.g., 2% sucrose). Characterize the microparticle with scanning electron microscopy (SEM), differential scanning calorimetry (DSC) and X-Ray diffraction (XRD).

1. A compound of formula I: 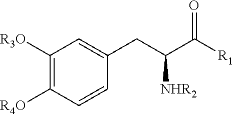
or a pharmaceutically acceptable salt, hydrate, and/or solvate thereof, wherein:
R₁ is a pharmaceutically acceptable polymeric moiety comprising a pharmaceutically acceptable polymer chain such that the carbonyl group is linked to R₁ through an ester, amide, carbonate or carbamate bond;
R₂ is hydrogen, or —(C═O)R₅ wherein R₅ is a C₁₋₃ straight or branched chain alkyl group; and
R₃ and R₄ are independently selected from hydrogen, C₁₋₃ straight or branched chain alkyl group, or —(C═O)R₆ wherein R₆ is —(O—CH₂—CH₂)ₙ—OCH₃ or a C₁₋₃ straight or branched chain alkyl group, and n is 1 to 5.

2. The compound of claim 2, wherein the pharmaceutically acceptable polymer chain comprising the polymeric moiety R₁ is selected from the group consisting of polyethylene glycol, poly(glycolide), poly(lactide), poly(caprolactone), poly(lactide-co-caprolactone), poly(lactide-co-glycolide), and poly(lactic acid)-butanol, poly(vinyl pyrrolidone), poly(vinyl alcohol), poly(ethyleneimine), poly(malic acid), poly(L-lysine), poly(L-glutamic acid), and poly ((N-hydroxyalkyl)glutamine), dextrins, hydroxyethylstarch, polysialic acid, polyacetals, N-(2-hydroxypropyl)methacrylamide copolymer, poly(amido amine) dendrimers, and mixtures, combinations and copolymers thereof.

3. A composition comprising a pharmaceutically effective amount of the compound of claim 1 and one or more pharmaceutically acceptable carriers or excipients.

4. The composition of claim 3, wherein the composition is injectable, inhalable, or topical.

5. The composition according to claim 3, wherein the composition is in the form of liposomes or micelles.

6. The composition according to claim 3, wherein the pharmaceutically acceptable carrier is castor oil or a derivative thereof.

7. A pharmaceutical composition comprising micro or nano particles comprising:
a pharmaceutically effective amount of the compound of formula I; and
a second pharmaceutically acceptable polymer,
wherein the compound of formula I is encapsulated in the second pharmaceutically acceptable polymer.

8. The pharmaceutical composition according to claim 7, wherein the second pharmaceutically acceptable polymer is selected from the group consisting of polyethylene glycol, poly(glycolide), poly(lactide), poly(caprolactone), poly(lactide-co-caprolactone), poly(lactide-co-glycolide), and poly(lactic acid)-butanol, poly(vinyl pyrrolidone), poly(vinyl alcohol), poly(ethyleneimine), poly(malic acid), poly(L-lysine), poly(L-glutamic acid), and poly ((N-hydroxyalkyl)glutamine), dextrins, hydroxyethylstarch, polysialic acid, polyacetals, N-(2-hydroxypropyl)methacrylamide copolymer, poly(amido amine) dendrimers, and mixtures, combinations and copolymers thereof.

9. The pharmaceutical composition according to claim 8, wherein the composition further comprises one or more pharmaceutically acceptable carriers or excipients.

10. The composition of claim 9, wherein the composition is injectable, inhalable, or topical.

11. A method for treating Parkinson's disease comprising administering the composition according to claim 3.

12. The method according to claim 11, wherein the composition is administered intravenously, intramuscularly, intraperitoneally, or subcutaneously.

13. The method according to claim 11, wherein the composition is co-administered with carbidopa and/or entacapone.

14. The method according to claim 12, wherein the composition is administered once daily.

15. The method according to claim 12, wherein the composition is administered twice weekly, thrice weekly, once weekly or biweekly.

16. The method according to claim 12, wherein the composition is administered once monthly.

17. A method for treating Parkinson's disease comprising administering the composition-according to claim 7.

18. The method according to claim 17, wherein the composition is co-administered with carbidopa and/or entacapone.

19. The method according to claim 17, wherein the composition is administered intravenously, intramuscularly, intraperitoneally, or subcutaneously.

20. The method according to claim 17, wherein the composition is administered once daily, twice weekly, thrice weekly, once weekly, biweekly, or once monthly.

21-23. (canceled)
